SEQUENCE LISTING (25 sequences)

SEQ ID NO: 1 - DNA, 1841 bp, Human
accgcgtccgccccgcgagcacagagcctcgcctttgccgatccgccgcccgtccacacccgccgccagctcaccatggatgatgatatcgccgcgctcgtcgtcgacaacggctccggcatgtgcaaggccggcttcgcgggcgacgatgccccccgggccgtcttcccctccatcgtggggcgccccaggcaccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtgcctcccgaggagcaccccgtgctgctgaccgaggcccccctgaaccccaaggccaaccgcgagaagatgacccagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaagatcattgctcctcctgagcgcaagtactccgtgtggatcggcggctccatcctggcctcgctgtccaccttccagcagatgtggatcagcaagcaggagtatgacgagtccggcccctccatcgtccaccgcaaatgcttctaggcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcattgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttcccccttttttgtcccccaacttgagatgtatgaaggcttttggtctccctgggagtgggtggaggcagccagggcttacctgtacactgacttgagaccagttgaataaaagtgcacaccttaaaaaaaaaaaaaaaaaaaaaaaaaaaaaaaaaaaaaaaaaaaaaa

SEQ ID NO: 2 - DNA, 1283 bp, Human
ctctctgctcctcctgttcgacagtcagccgcatcttcttttgcgtcgccagccgagccacatcgctcagacaccatggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgacaacgaatttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaacc

SEQ ID NO: 3 - DNA, 3221 bp, Virus
gaattccactgccttccaccaaactctgcaggatcccagagtcaggggtctgtatcttcctgctggtggctccagttcaggaacagtaaaccctgctccgaatattgcctctcacatctcgtcaatctccgcgaggactggggaccctgtgacgaacatggagaacatcacatcaggattcctaggacccctgctcgtgttacaggcggggtttttcttgttgacaagaatcctcacaataccgcagagtctagactcgtggtggacttctctcaattttctagggggatctcccgtgtgtcttggccaaaattcgcagtccccaacctccaatcactcaccaacctcctgtcctccaatttgtcctggttatcgctggatgtgtctgcggcgttttatcatattcctcttcatcctgctgctatgcctcatcttcttattggttcttctggattatcaaggtatgttgcccgtttgtcctctaattccaggatcaacaacaaccagtacgggaccatgcaaaacctgcacgactcctgctcaaggcaactctatgtttccctcatgttgctgtacaaaacctacggatggaaattgcacctgtattcccatcccatcgtcctgggctttcgcaaaatacctatgggagtgggcctcagtccgtttctcttggctcagtttactagtgccatttgttcagtggttcgtagggctttcccccactgtttggctttcagctatatggatgatgtggtattgggggccaagtctgtacagcatcgtgagtccctttataccgctgttaccaattttcttttgtctctgggtatacatttaaaccctaacaaaacaaaaagatggggttattccctaaacttcatgggctacataattggaagttggggaactttgccacaggatcatattgtacaaaagatcaaacactgttttagaaaacttcctgttaacaggcctattgattggaaagtatgtcaaagaattgtgggtcttttgggctttgctgctccatttacacaatgtggatatcctgccttaatgcctttgtatgcatgtatacaagctaaacaggctttcactttctcgccaacttacaaggcctttctaagtaaacagtacatgaacctttaccccgttgctcggcaacggcctggtctgtgccaagtgtttgctgacgcaacccccactggctggggcttggccataggccatcagcgcatgcgtggaacctttgtggctcctctgccgatccatactgcggaactcctagccgcttgttttgctcgcagccggtctggagcaaagctcatcggaactgacaattctgtcgtcctctcgcggaaatatacatcgtttccatggctgctaggctgtactgccaactggatccttcgccggacgtcctttgtttacgtcccgtcggcgctgaatcccgcggacgacccctctcggggccgcttgggactctctcgtccccttctccgtctgccgttccagccgaccacggggcgcacctctctttacgcggtctccccgtctgtgccttctcatctgccggtccgtgtgcacttcgcttcacctctgcacgttgcatggagaccaccgtgaacgcccatcagatcctgcccaaggtcttacataagaggactcttggactcccagcaatgtcaacgaccgaccttgaggcctacttcaaagactgtgtgtttaaggactgggaggagctgggggaggagattaggttaaaggtctttgtattaggaggctgtaggcacaaattggtctgcgcaccagcaccatgcaactttttcacctctgcctaatcatctcttgtacatgtcccactgttcaagcctccaagctgtgccttgggtggctttggggcatggacattgacccttataaagaatttggagctactgtggagttactctcgtttttgccttctgacttctttccttccgtcagagatctcctagacaccgcctcagctctgtatcgagaagccttagagtctcctgagcattgctcacctcaccatactgcactcaggcaagccattctctgctggggggaattgatgactctagctacctgggtgggtaataatttggaagatccagcatctagggatcttgtagtaaattatgttaatactaacgtgggtttaaagatcaggcaactattgtggtttcatatatcttgccttacttttggaagagagactgtacttgaatatttggtctctttcggagtgtggattcgcactcctccagcctatagaccaccaaatgcccctatcttatcaacacttccggaaactactgttgttagacgacgggaccgaggcaggtcccctagaagaagaactccctcgcctcgcagacgcagatctccatcgccgcgtcgcagaagatctcaatctcgggaatctcaatgttagtattccttggactcataaggtgggaaactttacggggctttattcctctacagtacctatctttaatcctgaatggcaaactccttcctttcctaagattcatttacaagaggacattattaataggtgtcaacaatttgtgggccctctcactgtaaatgaaaagagaagattgaaattaattatgcctgctagattctatcctacccacactaaatatttgcccttagacaaaggaattaaaccttattatccagatcaggtagttaatcattacttccaaaccagacattatttacatactctttggaaggctggtattctatataagcgggaaaccacacgtagcgcatcattttgcgggtcaccatattcttgggaacaagagctacagcatgggaggttggtcatcaaaacctcgcaaaggcatggggacgaatctttctgttcccaatcctctgggattctttccccatcatcagttggaccctgcattcggagccaactcaaacaatccagattgggacttcaaccccgtcaaggacgactggccagcagccaaccaagtaggagtgggagcattcgggccaaggctcacccctccacacggcggtattttgggctcgagccctcaggctcagggcatattgaccacagtgtcaacaattcctcctcctgcctccaccaatcggcagtcaggaaggcagcctactcccatctctccacctctaagagacagtcatcctcaggccatgcagtg

SEQ ID NO: 4 - DNA, 25 bp, Artificial
  Primer
ctttcgtgtaaattatgtaatgcaa

SEQ ID NO: 5 - DNA, 27 bp, Artificial
  Primer
aaaataaaaaagtattaaggcgaagat

SEQ ID NO: 6 - DNA, 19 bp, Artificial
  Primer
tggacatccgcaaagacct

SEQ ID NO: 7 - DNA, 20 bp, Artificial
  Primer
agacagcactgtgttggcgt

SEQ ID NO: 8 - DNA, 13 bp, Artificial
  Primer
gggcatgggtcag

SEQ ID NO: 9 - DNA, 15 bp, Artificial
  Primer
cgcccacataggaat

SEQ ID NO: 10 - DNA, 12 bp, Artificial
  Primer
gcgctcgtcgtc

SEQ ID NO: 11 - DNA, 11 bp, Artificial
  Primer
cggagccgttg

SEQ ID NO: 12 - DNA, 23 bp, Artificial
  Primer
aaatgcttctaggcggactatga

SEQ ID NO: 13 - DNA, 23 bp, Artificial
  Primer
aaacaaataaagccatgccaatc

SEQ ID NO: 14 - DNA, 24 bp, Artificial
  Primer
acttagttgcgttacaccctttct

SEQ ID NO: 15 - DNA, 25 bp, Artificial
  Primer
cgttccagtttttaaatcctgagtc

SEQ ID NO: 16 - DNA, 23 bp, Artificial
  Primer
cctcttcatcctgctgctatgcc

SEQ ID NO: 17 - DNA, 23 bp, Artificial
  Primer
ggggaaagccctacgaaccactg

SEQ ID NO: 18 - DNA, 23 bp, Artificial
  Primer
tcaaggtatgttgcccgtttgtc

SEQ ID NO: 19 - DNA, 21 bp, Artificial
  Primer
cgaaccactgaacaaatggca

SEQ ID NO: 20 - DNA, 25 bp, Artificial
  Primer
ctttcgtgtaaattatgtaatgcaa

SEQ ID NO: 21 - DNA, 25 bp, Artificial
  Primer
ctttcgtgttaaataagttatccaa

SEQ ID NO: 22 - DNA, 25 bp, Artificial
  Primer
ctttcgtgtaatttatgtaatgcta

SEQ ID NO: 23 - DNA, 27 bp, Artificial
  Primer
aaaataaaaaagtattaaggcgaagat

SEQ ID NO: 24 - DNA, 27 bp, Artificial
  Primer
aaaataaaaaagtattaaggcgatgaa

SEQ ID NO: 25 - DNA, 27 bp, Artificial
  Primer
tatataaaaaagtattaacgcgaacat

(19) United States
(12) Patent Application Publication
(10) Pub. No.: US 2006/0063175 A1
(43) Pub. Date: Mar. 23, 2006

(54) Method of polymerase chain reaction with ultra-low denaturing temperatures and applications thereof

(76) Inventors: Dingbang Xu, Shanghai (CN); Wenhui Xu, Shanghai (CN); Defen Zhu, Shanghai (CN); Wenkai Xie, Shanghai (CN)

Correspondence Address: ANNA WU, P.O. BOX 93286, CITY OF INDUSTRY, CA, 91715, US

(21) Appl. No.: 11/158,212

(22) Filed: Jun. 15, 2005

(30) Foreign Application Priority Data
Dec. 18, 2002 (CN) 02155183.9
Dec. 18, 2002 (CN) 02155184.7
Dec. 15, 2003 (WO) PCT/CN03/01063

(51) Int. Cl. C12Q 1/68; C12P 19/34

(52) U.S. Cl. 435/6; 435/91.2

(57) ABSTRACT

The invention relates to a method polymerase chain reaction (PCR) and the application thereof A method of PCR performed at ultra-low denaturing temperatures is provided. The denaturing temperatures of the templates adopted are 93-98° C. in the primary 2-3 cycles, and 60-87° C. in the follow-up cycles, those are much lower than 94-96° C., the conventional denaturing temperatures. It is found in the experiment that this method could not only become a universally applied PCR, but also control the reaction specificity by the template selection at ultra-low temperatures. The method possesses unique functions in excluding non-specific amplified products and false-negative results, excluding false-positivity brought about by the contaminants in products and discriminating genomic DNA from cDNA.

### TECHNICAL FIELD

The invention relates to molecular biology techniques, in particular, relates to a method of polymerase chain reaction and applications thereof

### TECHNICAL BACKGROUND

Polymerase chain reaction (PCR) is a high efficient method for amplifying special DNA. It is widely used in various medical fields, particularly in clinical diagnosis. PCR is mainly consisted of 25-35 cycles with a periodic change of temperature. Each cycle contains three steps: denaturing, annealing and extending. The denaturing step enables the melting of double-stranded DNAs of original templates or amplified products into two single strands, which bind complementarily to forward or reverse primers, respectively, at the annealing temperature, then followed by an extending step, so that a cycle is finished. The denaturing step can be considered as a beginning step for each cycle, and it is necessary for the whole amplification process.

The specificity and efficiency of PCR mainly depend on the annealing and extending steps. The studies of denaturing steps were much less than those of annealing and extending steps. It has been described, in various technical guides to PCR, that the common denaturing temperature was in the range of 94-95° C., which had been used in a majority of hundred thousands of publications regarding PCR applications. For denaturing, higher temperatures, such as 96° C., and lower temperatures, such as 90-94° C., have rarely been used. The lowest denaturing temperature reported in literatures was 87° C.

Up to date, the denaturing temperature has been defined to 94-95° C. It is reasonable, since this temperature is close to the limit at which DNA-polymerase can tolerate and sufficiently show its thermostability. On the other hand, at this temperature, almost all original templates and amplified products with different lengths can be melted completely, so that the whole amplification process can be finished. For such reasons, there is no suspicion over the past ten years, in the necessity and reasonableness of widely using such high denaturing temperatures, and no one wonders whether a great adjustment to the denaturing temperature is capable.

The upper limit of denaturing temperatures is restricted by thermostability of DNA polymerase. The half-life of DNA polymerase decreases along with the increase of the denaturing temperature, and quickly drops down at over 90° C. Taking the widely used and extremely thermostable Taq DNA polymerase for instance, the half-life thereof is about 130, 40 or 5 min., respectively, at the temperatures of 92.5° C., 95° C. or 97.5° C. In general, the denaturing period is 30 sec. for each cycle. It is obvious that the activity of Taq enzyme will drop down significantly after several cycles and it is hard to finish the amplification process of about 30 cycles if the denaturing temperature is over 97° C.

The lower limit of denaturing temperatures is restricted by melting temperatures (Tm) of both original template DNAs and amplified products. In general, the length of PCR amplified products are 150-800 bases, and the Tm is in the range of 85-92° C. in a standard PCR reaction solution. The double strands of the original templates or amplified products can not be melted and the amplified process can not be finished if the denaturing temperature is lower.

For enabling DNA polymerase with some good characters but less thermostability to be used in PCR, a method was developed in the beginning of 1990s. In this method, chemical denaturants in high concentrations, for example, 10-50 % forinamide, 40 % glycerin or 5.5 M proline, etc., were added into the PCR reaction solution(Nucleic Acid Research, 1999, Vol. 27 (6): 1566-1568). The high concentrations of denaturants result in a significant decrease of DNA Tm, so the denaturing step can be completed at about 70° C. However, the high concentrations of denaturants, cause a substantial decrease of Tm for all DNAs, including original templates, amplified products of interest, non-specific amplified products and primers, thus decreasing the annealing and extending temperatures accordingly and significantly. Moreover, the high concentrations of chemical denaturants not only inhibit the activity of DNA polymerase, but also result in changes of the density, viscosity and thermoconductivity of the PCR reaction solution, thus exerting remarkable and complicated negative impacts on the specificity and efficiency of the PCR reaction. There is no interesting and substantial advantage of this method except for the application of the less thermostable DNA polymerase in PCR. Since the continuous appearance of various DNA polymerases with better characteristics and higher temperature tolerance in recent years, the method of using chemical denaturants in high concentrations to decrease the denaturing temperature was not widely spread in either scientific researches or clinical detection.

Based on the review of the principle of PCR and the analysis of action and mechanism of the denaturing step, the inventors have revised and improved the traditional process for PCR, extended the denaturing temperature range of PCR and decreased it to 60-87° C., thus resulting in particular technical effects.

### DISCLOSURE OF INVENTION

Technical Problem to be Solved

The technical problem to be solved in the invention is to provide a method of polymerase chain reaction with an ultra-low denaturing temperature and the application thereof, so as to make a breakthough of the conventional denaturing temperature of the template in PCR and overcome the following defects of current PCR: the impossibility of eliminating non-specific amplified products by adjusting denaturing temperatures, of recognizing false positive results due to the contamination of amplified products, and of eliminating false negative results.

Inventive Concept

The principle and concept of the invention are concluded as follows:

First, the denaturing temperature still remains at 94-98° C. for the first template denaturing and primary two cycles. After the first denaturation and the denaturing step of the first cycle, the original template containing amplified products of interest is melted into two full-length single strands. These single strands are annealed with primers and extended to become two pairs of non-intact double strands after the end of the first cycle. Each pair of double strands consists of one full-length original template strand and one half-length amplified strand. During the denaturing step of the second cycle, these two pairs of non-intact double strands are melted to four single strands. They are annealed with the primers and extended to become four pairs of non-intact double strands, wherein two pairs are identical to the products of first cycle, and the other two consist of half-length amplified strands and amplified strands of interest. The original template and the “half-amplified products” obtained in the first cycle usually have large molecular weights, and the melting temperatures thereof are higher and hard to be estimated and detected. It is allowed to melt the most original templates and half-amplified products at the current denaturing temperatures, 94-95° C. In view of the fact that the half-life of Taq DNA polymerase at 97.5° C. is still 5 min, the first denaturing temperature and the denaturing temperatures of the primary two cycles can be expanded to 94-98° C. By this way, the original templates and “half-amplified products” can be quickly and sufficiently melted. The denaturing time can be shortened to 1-15 sec. when the denaturing temperature is higher than 96° C.

Second, ultra-low denaturing temperatures of 60-87° C. were adopted after primary two cycles. After the formation of amplified products of interest in the first round, these single-stranded DNAs will take part in the amplification process as templates so that products will cumulate continuously in an exponential way. The original templates and the “half-amplified products” can still participate in the amplification process as templates, but with the performance of the PCR process, their action in the accumulation of amplified products of interest is getting less and less. So the denaturing temperatures of the rest PCR cycles merely need to satisfy the melting of the amplified products of interest and it is unnecessary to consider the denature of the original templates and half-amplified products. Whereas, the Tm of the amplified products of interest can be estimated, calculated or detected by an experiment based on the length, the composition of bases and the sequences of the amplified products.

Third, the principle of eliminating non-specific products in the PCR reaction at ultra-low temperatures was provided. The human genome contains about 3 billions of base pairs in all, whereas, the type of permutation and combination of nucleotides with eighteen bases reaches 70 billions(4¹⁸), hence the probability of emergence of the DNA fragment entirely matched the sequence of the primer of 18 bases is little. However, even if primers of 25 bases or much longer are adopted, non-specific amplified products still often appear. This is because that although it hardly exists any other DNA fragment matching entirely with both forward and reverse primers in the samples, except the gene of interest, it may exists some or many DNA fragments matching at a higher degree with both forward and reverse primers. The primers can even combine with seriously mismatched DNA fragments at not very precise annealing temperatures, resulting in the formation of non-specific products. The upstream and downstream sequences of non-specific amplified products formed in the first round in the primary two cycles have entirely complemented with the primers, respectively. At this time, the continuous amplification of non-specific products can not be prevented even if the annealing temperature is raised. If the original template concentration of non-specific products is higher or the length of non-specific products is shorter, the amplification efficiency of non-specific products is likely higher than that of the amplified products of interest, resulting in the regnant position of the non-specific products in the amplified products. When the denaturing temperature is between 94-95° C., almost all the non-specific products can be melt, then the denaturing step is finished and the cycles continues. If the temperature of denaturing step is limited to a degree somewhat higher than the melting temperature of the amplified product of interest, those non-specific amplified products with higher Tm will abort because of the failure of finishing the melting process.

Fourth, when the lengths of the products are less than 400 bases, it is an absolutely feasible project for choosing the products of interest with denaturing temperatures lower than 87° C. The lengths of PCR amplified products are usually in a range of 150-800 bases. It has been shown in the present research that, within this range, especially in a range of 150-400 bases, there were biggish differences in the Tm of DNA due to the differences of the composition and arrangement of bases. Taking the amplified products with a length of 200 bases for instance, the lowest melting temperature of the DNA fragment amongst the randomly chosen 22 DNA fragments is 77.2° C., which is 8.7° C. lower than the average melting temperature and 16.4° C. lower than the highest one. Therefore, in this range of length of bases, amplified products of interest with lower melting temperatures will be chosen for every DNA fragment with a certain length. The lower the melting temperatures of chosen amplified products of interest are, the more the non-specific amplified products are aborted by controlling the temperature of the denaturing step.

Fifth, when the length of the product is less than 150 bases, the denaturing temperature will decrease greatly. There is not any restriction towards the length of PCR products in many applications of PCR, such as the gene expression analysis and the virus infection detection, in which short amplified products with length less than 150 bases can be adopted. When the length of amplified product is less than 150 bases, especially, less than 70 bases, the average melting temperature of DNAs will decrease greatly along with the decrease of the length. Meanwhile, the difference between the highest and lowest melting temperatures will increase along with the decrease of the length of strand. Thus, the selection of a PCR method with ultra-low denaturing temperatures with short amplified products has more potential and special advantages. For example, when the lengths of amplified products are less than 200, 120 and 70 bases, respectively, the lowest melting temperature of amplified products may be lower than 80, 75 and 70° C., respectively. The denaturing step can be accomplished at a temperature a little higher than 80, 75 or 70° C.

Sixth, the denaturing temperature should be taken as one of the criteria applied to the selection of suitable primers. Lengths of amplified products and all characteristics, including melting temperatures, depend on primer pairs. Melting temperatures of amplified products have never been taken as one of the criteria applied to the selection of primers in up to date researches. Melting temperatures of amplified products have never been taken as one of the criteria applied to the judgement of the preciseness of primers in all kinds of primer designing softwares, either. However, even if ordinary primer designing softwares are adopted, i.e. in the case of not considering the contribution of the preciseness of primers brought about by melting temperatures of amplified products, it appears a given proportional primers within the high preciseness primers searched by primer designing softwares automatically. The melting temperature of the amplified product obtained by using such a primer is remarkably lower than the average temperature of amplified products with such a length, thus the method of PCR at low denaturing temperatures is valuable and widely feasible as well.

Seventh, when the lengths of amplified products are in the range of 400-1,000 bases, primers causing the denaturing temperature of amplified products lower than 87° C. can still be selected, and non-specific products can be eliminated in the relevant PCR reaction. Average, lowest and highest melting temperatures of 22 amplified products with lengths from 30 to 2,000 bases were detected by using the same method. It is shown that when lengths of amplified products are 30-200 bases, the lowest melting temperature is about 16-30° C. lower than the highest one. It is shown, from the distribution of melting temperatures of DNA fragments with different lengths, that, if lengths of amplified products are within a range of 400-1,000 bases, it is very easy to select some primer pairs the melting temperatures of amplified products thereof a little higher than 80° C. At relevant denaturing temperatures, denaturing steps will not be finished for most of non-specific products with lengths longer than 400 bases and part of non-specific amplified products with lengths less than 400 bases and they will abort due to the higher melting temperature. If there does not exist any predetermined limitation, it is very easy to find primers which result in the length of the amplified product of interest less than 200 bases and the melting temperature thereof a little higher than 75° C. The adoption of relevant denaturing temperatures can enable the abortion of non-specific amplified products with different lengths. If the length of amplified product of interest is less than 70 bases, it is very easy to find primers that result in the melting temperature of the amplified product close to 70° C., even lower than 70° C. In this case, non-specific amplified products can almost be eliminated completely. Of course, it always exists some DNA fragments with lengths of about 70 bases among several billion base pairs in human genome, the melting temperatures thereof are also about 70° C. It is imaginable that the probability of highly matching with two primers among these fragments at a time is very little.

Eighth, there is less possibility of non-specific products that two primers can both anneal with in a short distance, so the PCR method with an ultra-low denaturing temperature can efficiently control the formation of non-specific amplified products. Each cycle of PCR includes three steps, denaturing, annealing and extending, and none of them is dispensable. The denaturing temperature adopted in the standard PCR method is in a range of 94-95° C., which is necessary for the sufficient melting of the original template DNAs in the sample. Generally, no matter whether genomic DNAs or complementary DNAs needs to be melted at 94-95° C. However, it is unnecessary for many amplified products to continue denaturing at 94-95° C. in the follow-up cycles since most of amplified products with less than 1,000 bases can denature at a temperature below 94° C. more often than not. According to the standard PCR method, all the amplified products formed in the primary three cycles, including amplified products of interest and non-specific amplified products, can continue to finish the denaturing, annealing and extending steps in the later cycles until the reaction is over. The melting temperatures of amplified products of the primers chosen in the invention are very low, and they can finish denaturing in the follow-up dozens of cycles at low denaturing temperatures. However, most of non-specific amplified products formed in the primary three cycles can not finish denaturing steps in the follow-up cycles because of their higher melting temperatures, thus resulting in the abortion of these non-specific ampliphied products. The shorter the lengths of selected amplified products of interest or the lower the selected melting temperatures are, the lower the feasible denaturing temperatures and the more the non-specific amplified products excluded by adjusting the denaturing temperature are. It brings about a chance to reduce the false negative results caused by the variability of the viruses and bacteria in clinical diagnosis. When an ultra-low denaturing temperature is applied in the PCR reaction, the amplification process can be finished at a temperature over ten degrees lower than the allowed highest annealing temperature without any interference of non-specific amplified products.

In order to explain the principle and method of the invention, human CyclinD1 gene bank serial number: NM_{—}053056) with over 4,000 bases was exemplified and some data, e.g., Tms of DNAs have been analyzed by virtue of primer designing sofware-Oligo. Each number is a statistic result of 22 calculated values (Table 1).

TABLE 1
Melting temperatures of DNAs with different lengths

| Lengths of DNAs | Average Tm | Lowest Tm | Highest Tm |
|---|---|---|---|
| (base pairs) | (° C.) | (° C.) | (° C.) |
| 30 | 73.4 | 58.9 | 87.6 |
| 40 | 77.4 | 64.1 | 88.7 |
| 50 | 79.3 | 66.4 | 91.0 |
| 60 | 80.7 | 68.6 | 91.8 |
| 70 | 82.0 | 69.6 | 91.8 |
| 80 | 82.6 | 70.8 | 91.9 |
| 90 | 82.8 | 72.3 | 89.1 |
| 100 | 83.3 | 73 | 91.9 |
| 120 | 84.2 | 74.8 | 91.9 |
| 150 | 84.8 | 76.3 | 93.8 |
| 200 | 85.9 | 76.3 | 93.8 |
| 400 | 87.0 | 80.5 | 93.4 |
| 600 | 87.3 | 82 | 92.7 |
| 800 | 87.4 | 83.1 | 92.2 |

Technical Solution

The method of polymerase chain reaction (PCR) with ultra-low denaturing temperatures of the invention includes steps in turn as follows: denaturing templates, annealing primers, extending and synthesizing complementary DNA strands by catalysis of DNA polymerase. Based on the premise of not adding any chemical denaturant, circular amplification reactions proceed in the three steps mentioned above. The denaturing temperature of the template is in the range of 93-98° C. in primary two or three cycles, and is changed into 60-87° C. in the follow-up cycles, preferably, 70-82° C.

The amplification reaction products with lengths of 24-1,000 bases, preferably, 40-150 bases, are adopted in the method of polymerase chain reaction with ultra-low denaturing temperatures according to the invention.

In case that the difference between the denaturing temperature of the original template and that of the product is 7-28° C., said PCR method with ultra-low denaturing temperatures is efficiently used to eliminate non-specific amplified products. Preferably, the difference between the denaturing temperature of the original template and that of the product is 10-20° C.

In case that the mismatching between the original template and the primer is 1-5 bases and the melting temperature of the product is 60-87° C., said PCR method with an ultra-low denaturing temperature is efficiently used to exclude false-negative results. Especially, in case that the mismatching between the original template and the primer is 1-3 bases, the effect of excluding false-negative results will be better. The annealing temperatures of primer and template adopted in the reaction are in the range of 32-65° C., preferably, 46-58° C.

Said PCR method with ultra-low denaturing temperatures can be used to detect whether there exists any contamination in the amplified products after the reaction of templates at denaturing temperatures of 94-95° C. and 68-87° C., respectively, in primary two cycles and the denaturation at 60-87° C. in the follow-up cycles.

Another application of said PCR method is to discriminate genomic DNAs and cDNAs. The approach is to perform two PCR reactions with template samples as follows:

(1) Performing primary two cycles at the denaturing temperature of 94-95° C., and follow-up cycles, 68-87° C.; and

(2) Performing primary one cycle at the denaturing temperature of 94-95° C., and follow-up cycles, 68-87° C.

Genomic DNAs can be melted at 94-95° C. but not be denatured at 68-87° C., so they need at least two high-temperature denaturing cycles for being completely amplified, while complementary DNAs (cDNAs) obtained from the reverse transcription of gene-specific reverse primers only need one high-temperature denaturing cycle for being completely amplified. Namely, genomic DNAs show positive results in reaction (1) and negative results in reaction (2), whereas cDNAs show both positive results in reactions (1) and (2).

One of the cruces for realizing the invention is to search excellent primers automatically by using a primer designing software or to determine primers artificially in following steps: selecting the areas of primers preliminarily by virtue of the Tm atlas of gene sequences of interest showed by the primer designing software; analyzing characteristics of candidate primers by using the software; and then making a decision. The former approach is suitable to the design of primers for amplified products having a length less than 100 bases. The latter approach is suitable to the design of primers for amplified products having a length over 100 bases.

Same as the common criterion of selecting primers in the standard PCR method, the primers selected in the invention should possess high preciseness, namely, a lower complementation of 3′ terminal bases, a less hairpin structure, a lower mismatching combination intensity and a higher specific combination intensity. The quite wide range of primer lengths is from 12 bases to 50 bases. The chief characteristic of the primers selected in the invention is that the melting temperature of amplified products thereof is in the range of 60-85° C., preferably, 72-80° C.

The method of decreasing denaturing temperatures of the invention is different from that of adding a chemical denaturant in the principle, but compatible in applications. The addition of a chemical denaturant in a lower concentration, if desired, may further decrease the range of denaturing temperatures, and retain advantages of the invention at the same time.

All the parameters in the PCR reaction of the invention are basically identical to those in the standard PCR. The number of cycles is generally from 20 to 45. The denaturing temperature in the primary 2 or 3 cycles of PCR reaction is from 94° C. to 95° C. Primary amplified products of interest have been formed during these cycles at a conventional denaturing temperature. The distinction of the invention is that the denaturing temperatures in the follow-up 2045 cycles are decreased to 60-87° C. The temperature which is actually performed depends on the melting temperature of the amplified product of interest. The temperature in the denaturing step should be a little higher than this temperature. The closer the denaturing temperature to the melting temperature is, the better the specificity of the PCR reaction is.

The DNA template adopted in the invention is cDNA which is prepared from human muscle tissue total RNA (from Clontech Company) by using a cDNA preparation kit (Advantage RT for PCR kit, Clontech Company). The primers used are Oligo(dT) ₁₈in the kit. The volume in the PCR is 10 μl, and each tube is added with 1 μl 1 μg/1λ RNA.

Advantage 2 kits of Clontech Company are used in all the PCR reactions of the invention. The final concentration of primers is 0.5 μM.

Beneficial Effects

The PCR method with ultra-low denaturing temperatures not only improves and exerts the characteristics of the conventional PCR method, such as the simplicity, speediness, specificity and sensitivity, but also expands functions and applications of the PCR method.

Another special advantage of the PCR method with ultra-low denaturing temperatures is to greatly shorten the PCR reaction time. First, the denaturing temperature in the invention is 60-87° C., preferably, 72-82° C. Moreover, the annealing temperature range is the same as that of the standard PCR. Taking the annealing temperature of 55° C. and denaturing temperature of 75° C. for instance, the difference of temperature in each cycle is 20° C. In comparison with the prior difference of temperature of 40° C. (95 minus 45), half of the ramp time is saved. Second, the denaturing temperature in the invention is controlled to be a little higher than the melting temperature of the amplified product of interest. In case of small reaction volume and rapid thermal conduction, the denaturing time only needs 1 sec, being dozens folds saved in comparison with the general time of 15 sec to 1 min. Third, most of the amplified products obtained from the PCR method with ultra-low denaturing temperatures are short, so that the amplification process can be finished in the ramp and the extending time of 1 sec. This also saves dozens folds of time in comparison with the general extending time of 1 min. In all examples of the invention, the PCR reaction with 30 cycles can be finished in 15 min, whereas it generally needs one hour to one and half an hour in the standard PCR method.

Except the primary three cycles, the denaturing temperature in other cycles of the PCR method with ultra-low denaturing temperatures of the invention is lower than 87° C. It is obvious that the activity of Taq polyerase remains stable before and after the reaction. This is in favor of performing PCR circularly and also in favor of keeping the stability of the whole reaction.

In aspect of application, it is shown, in the analysis on the mechanism of PCR process and our practical experiments, that the PCR method with ultra-low temperatures possesses substantial advantages. This method can effectively prevent or thoroughly exclude the formation of non-specific products by two kinds of mechanisms, controlling the annealing temperature and the denaturing temperature. However, the formation of non-specific products can be found everywhere in the standard PCR method, and it brings about troubles to PCR researchers and clinical analysts. The following experimental examples indicate that the method of ultra-low denaturing temperature can effectively control the formation of non-specific products. In these examples, even very low annealing temperatures were adopted, i.e., in the case of unloosing the control of annealing temperatures, not any non-specific product formed.

### SPECIFIC EMBODIMENTS

### EXAMPLE 1

The Feasibility of Primer Designing of the PCR with Ultra-Low Denaturing Temperatures

Taking the hepatitis virus B gene, human actomyosin gene and human glyceraldehyde-3-phosphate dehydrogenase gene for instance, we used the primer designing software-Oligo to test the frequency of the appearance of primers of interest which can amplify short products with lengths less than 100 bases. It was shown in the results that, for each length of each testing gene, tens to hundreds pairs of excellent primers with high preciseness could be found, moreover, about 30-80% of the primers produced amplified products having melting temperatures lower than 80° C. The test results of the human actomyosin gene are shown in Table 2.

TABLE 2
Number of excellent primer pairs found in human actomyosin gene

| Lengths of | Tm of amplified products (° C.) | | | | |
|---|---|---|---|---|---|
| amplified products | <70 | 70.1-75 | 75.1-80 | 80.1-85 | >85 |
| 31-40 | 2 | 32 | 50 | 16 | 0 |
| 41-50 | 0 | 27 | 36 | 38 | 0 |
| 51-60 | 0 | 0 | 47 | 50 | 3 |
| 61-70 | 0 | 0 | 40 | 27 | 33 |
| 71-80 | 0 | 0 | 30 | 13 | 56 |
| 81-90 | 0 | 0 | 51 | 37 | 13 |
| 91-100 | 0 | 0 | 45 | 33 | 22 |

It is shown in Table 2 that the PCR method with ultra-low denaturing temperatures is commonly suitable to the amplification of products having lengths less than 100 bases and partly suitable to those products having lengths of 100-150 bases. The average melting temperatures of products is between 83-87° C., and the difference between the highest and lowest melting temperature is over 10° C. It is suggested from the above that it is feasible to find some primers which produce amplified products having melting temperatures lower than 80° C.

### EXAMPLE 2

Primer Designing of PCR with Ultra-Low Denaturing Temperatures

Taking the full gene sequence of human actomyosin gene for instance, the following primers were designed by using primer designing software-Oligo (see table 3 for designing results).

TABLE 3

Taking the human actomyosin gene sequence
as an example to design primers of PCR
with ultra-low denaturing temperatures

| | | Length | Tm of | Length | Tm of |
|---|---|---|---|---|---|
| | | of | Primer | of | Product |
| Primer | Sequence 5′-3′ | Primer | (° C.) | Product | (° C.) |
| 9A1 5′ | CTT TCG TGT AAA TTA TGT AAT GCA A | 25 | 65.8 | 62 | 67.9 |
| 9A1 3′ | AAA ATA AAA AAG TAT TAA GGC GAA GAT | 27 | 66.0 | | |
| 9A2 5′ | TGG ACA TCC GCA AAG ACC T | 19 | 65.4 | 41 | 77.2 |
| 9A2 3′ | AGA CAG CAC TGT GTT GGC GT | 20 | 65.7 | | |
| 9A3 5′ | GGG CAT GGG TCA G | 13 | 47.9 | 32 | 76.1 |
| 9A3 3′ | CGC CCA CAT AGG AAT | 15 | 52.1 | | |
| 9A4 5′ | GCG CTC GTC GTC | 12 | 45.3 | 25 | 76.9 |
| 9A4 3′ | CGG AGC CGT TG | 11 | 41.9 | | |
| 9A5 5′ | AAA TGC TTC TAG GCG GAC TAT GA | 23 | 69.7 | 103 | 78.8 |
| 9A5 3′ | AAA CAA ATA AAG CCA TGC CAA TC | 23 | 69.6 | | |
| 9A6 5′ | ACT TAG TTG CGT TAC ACC CTT TCT | 24 | 68.0 | 144 | 77.5 |
| 9A6 3′ | CGT TCC AGT TTT TAA ATC CTG AGT C | 25 | 69.7 | | |

### EXAMPLE 3

PCR Reaction with Ultra-Low Denaturing Using the Primer Pairs Designed in Example 2

Reaction conditions: denaturing at 95° C. for 60 sec, annealing and extending at 62° C. (for 9A1, 9A2, 9A5 and 9A6) or 45° C. (for 9A3 and 9A4) for 5 sec, 2 or 3 cycles; then denaturing at 68-82° C. (for 5 sec, annealing and extending at 62° (for 9A1, 9A2, 9A5 and 9A6) or 45° C. (9A3 and 9A4) for 5 sec, 25 follow-up cycles in all (see Table 4).

TABLE 4
Results of PCR reaction with ultra-low denaturing temperature

| | | Primary | | | | | | | | | | | |
|---|---|---|---|---|---|---|---|---|---|---|---|---|---|
| Primer | Length of | cycle | Denaturing temperature of the follow-up cycles (° C.) | | | | | | | | | | |
| pairs | product | numbers | 68 | 70 | 72 | 73.7 | 74.9 | 76.4 | 78.1 | 79.5 | 80.5 | 81.3 | 82 |
| 9A1 | 62 | 3 | ± | + | + | + | + | + | + | + | + | + | + |
| 9A2 | 41 | 2 | − | − | − | − | − | − | + | + | + | + | + |
| 9A3 | 32 | 2 | − | − | − | − | − | − | + | + | + | + | + |
| 9A4 | 25 | 3 | − | − | − | − | − | ± | + | + | + | + | + |
| 9A5 | 103 | 2 | − | − | − | − | − | − | − | + | + | + | + |
| 9A6 | 144 | 3 | − | − | − | − | − | − | − | + | + | + | + |

+ indicates positive results;
− indicates negative results;
± indicates weak positive results.

### EXAMPLE 4

Application of the PCR Method with Ultra-Low Denaturing Temperature in Excluding Non-Specific Products

Optionally selected two pairs of HBV primers with normal lengths by using primer designing software-Oligo are as follows: (5′-3′)

| | |
|---|---|
| 9A8 5′: | CCT CTT CAT CCT GCT GCT ATG CC |

| Tm of the product: 85.5° C. | |
|---|---|
| 9A8 3′: | GGG GAA AGC CCT ACG AAC CAC TG |
| Length of the product: 315 | |
| 9A9 5′: | TCA AGG TAT GTT GCC CGT TTG TC |
| Tm of the product: 84.1° C. | |
| 9A9 3′: | CGA ACC ACT GAA CAA ATG GCA |

Length of the product: 251

Primers 9A8, 9A9 and 9A1-9A6 were reacted under the following conditions: denaturing at 95° C. for 60 sec, annealing at 45° C. for 15 sec, and extending at 62° C. for 15 sec, for 2 or 3 cycles; then denaturing at 85° C. for 5 sec, annealing at 45° C. for 15 sec, and extending at 62° C. for 15 sec, for 25 follow-up cycles in all.

It was shown that both reactions of 9A8 and 9A9 at the conventional denaturing temperature, under the low-temperature annealing condition, produced a lot of non-specific products without normal products with lengths of 315 and 251 bp. In contrast, normal products with expected lengths can still be obtained by using primers of 9A1-9A6.

### EXAMPLE 5

Application of PCR with Ultra-Low Denaturing Temperatures in Excluding False-Negative Results

It is shown both in the analysis of performances of pre-designed primer pairs of human actomyosin gene (Table 5, Table 6) by using primer analysis software-Oligo and the PCR reaction experiments (Table 7) that of the products of interest have still been amplified specifically even if the maximum mismatching is five between the primers and the template sequences of interest, namely, one primer can be used to deal with various variants containing several mutated sequences.

TABLE 5

Designed primer pairs of human actomyosin
gene sequence containing mismatching

| | | | |
|---|---|---|---|
| Matching | | | |
| degree | | Mis- | |
| with | | matching | |
| template | Name | numbers | Sequences (5′to 3′) |

| | | | | |
|---|---|---|---|---|
| Forward | Match | A 5′ | 0 | CTT TCG TGT AAA TTA |
| primers | absolutely | | | TGT AAT GCA A |
| | Mis- | AM1 5′ | 5 | CTT TCG TGT TAA ATA |
| | matching | | | AGT TAT CCA A |
| | exists | AM3 5′ | 2 | CTT TCG TGT AAT TTA |
| | | | | TGT AAT GCT A |
| Reverse | Match | A 3′ | 0 | AAA ATA AAA AAG TAT |
| Primers | absolutely | | | TAA GGC GAA GAT |
| | Mis- | AM2 3′ | 2 | AAA ATA AAA AAG TAT |
| | matching | | | TAA GGC GAT GAA |
| | exist | AM4 3′ | 3 | TAT ATA AAA AAG TAT |
| | | | | TAA CGC GAA CAT |

TABLE 6
Analysis on performances of primer pairs of human actomyosin
gene by using primer analysis software-Oligo

| Name | Numbers of | Mismatching | Approximate | Specific binding |
|---|---|---|---|---|
| of | mismatching | positions | melting | intensity with |
| primers | bases | (from 3′) | temperature | templates |
| A 5′ | 0 | | 64.6° C. | 395 points |
| AM1 5′ | 5 | 4, 7, 10, 13, 16 | 39.3° C. | 165 points |
| AM3 5′ | 2 | 2, 14 | 52.5° C. | 192 points |
| A 3′ | 0 | | 66.4° C. | 480 points |
| AM2 3′ | 2 | 1, 4 | 58.6° C. | 201 points |
| AM4 3′ | 4 | 3, 9, 25, 27 | 46.8° C. | 248 points |

TABLE 7
The test results of PCR reaction with an ultra-low denaturing temperature (72° C.) of
human actomyosin primer pairs at different annealing and extending temperatures

| PCR | | | | | | | | | | | |
|---|---|---|---|---|---|---|---|---|---|---|---|
| positive | Forward | Reverse | Annealing and extending temperature ° C. | | | | | | | | |
| results | primers | primers | 46 | 49.2 | 51.5 | 54.4 | 57.8 | 60.6 | 62.8 | 64.4 | 66 |
| 1 | A5′ | A 3′ | Yes | Yes | Yes | Yes | Yes | Yes | Yes | Yes | No |
| 2 | A5′ | AM2 3′ | Yes | Yes | Yes | Yes | Yes | No | No | No | No |
| 3 | A5′ | AM4 3′ | Yes | Yes | Yes | weak | No | No | No | No | No |
| 4 | AM3 5′ | A 3′ | Yes | Yes | Yes | Yes | Yes | weak | weak | No | No |
| 5 | AM3 5′ | AM2 3′ | Yes | Yes | Yes | No | No | No | No | No | No |
| 6 | AM3 5′ | AM4 3′ | Yes | weak | No | No | No | No | No | No | No |
| PCR | | | | | | | | | | | |
| positive | Forward | Reverse | | | | | | | | | |
| results | primers | primers | 32.0 | 34.3 | 35.9 | 37.9 | 40.3 | 42.3 | 43.8 | 44.9 | 46.0 |
| 7 | AM1 5′ | A 3′ | Yes | Yes | Yes | No | No | No | No | No | No |
| 8 | AM1 5′ | AM2 3′ | No | No | No | No | No | No | No | No | No |
| 9 | AM1 5′ | AM4 3′ | Yes | Yes | No | No | No | No | No | No | No |

Reaction conditions: denaturing at 95° C. for 15 sec, annealing and extending at 46-66° C. or 32-46° C. for 15 sec, 3 cycles in all; then denaturing at 72° C. for 15 sec, annealing and extending at 46-66° C. or 32-46° C. for 15 sec, 25 cycles in all.

Conclusion:

(1) Absolutely matched primers can anneal and finish the amplification process at about 65° C.

(2) When one primer can entirely match and the other primer has 1-4 mismatching, the specific combination intensity with template is within a range of 192-248 points, the annealing and amplification process can still be finished at about 54-58° C.

(3) When one primer can entirely match and the other primer has 5 mismatching, the specific combination intensity with template is 165 points, the annealing and amplification process can still be finished at about 36° C.

(4) When each primer of the primer pair has 1-5 mismatching, they all can finish amplification process, and the highest annealing temperature allowed is a little lower in comparison with the case that one primer is entire matching.

(5) In case of annealing at the above low temperatures, still only amplified products of interest are formed.

### EXAMPLE 6

Application in the Detection of Contamination of Amplified Product Fragments at Different Denaturing Temperatures

Taking the full gene sequence of human actomyosin gene for instance, the following primers were designed by using primer designing software-Oligo (see table 3 for designing results).

TABLE 8

Taking the human actomyosin gene sequence
as an example to design relevant primers
of the products with super-short lengths

| Name | | Length | Tm of | Length | Tm of |
|---|---|---|---|---|---|
| of | | of | primer | of | product |
| primer | Sequence 5′-3′ | primer | (° C.) | product | (° C.) |
| 9A1 5′ | CTT TCG TGT AAA TTA TGT AAT GCA A | 25 | 65.8 | 62 | 67.9 |
| 9A1 3′ | AAA ATA AAA AAG TAT TAA GGC GAA | 27 | 66.0 | | |
| | GAT | | | | |
| 9A2 5′ | TGG ACA TCC GCA AAG ACC T | 19 | 65.4 | 41 | 77.2 |
| 9A2 3′ | AGA CAG CAC TGT GTT GGC GT | 20 | 65.7 | | |
| 9A3 5′ | GGG CAT GGG TCA G | 13 | 47.9 | 32 | 76.1 |
| 9A3 3′ | CGC CCA CAT AGG AAT | 15 | 52.1 | | |
| 9A4 5′ | GCG CTC GTC GTC | 12 | 45.3 | 25 | 76.9 |
| 9A4 3′ | CGG AGC CGT TG | 11 | 41.9 | | |
| 9A5 5′ | AAA TGC TTC TAG GCG GAC TAT GA | 23 | 69.7 | 103 | 78.8 |
| 9A5 3′ | AAA CAA ATA AAG CCA TGC CAA TC | 23 | 69.6 | | |
| 9A6 5′ | ACT TAG TTG CGT TAC ACC CTT TCT | 24 | 68.0 | 144 | 77.5 |
| 9A6 3′ | CGT TCC AGT TTT TAA ATC CTG AGT C | 25 | 69.7 | | |

Polymerase chain reaction was Performed with primer pairs 9A1-9A6, and relevant contaminated amplified fragments 10 ng, 1 ng, 100 pg, 10 pg, 1 pg, 100 fg, 10 fg were added to template. Template samples were treated at two denaturing temperatures of 95° C. and 79° C., respectively, for 15 sec, then were annealed and extended at 62° C. (for 9A1, 9A2, 9A5 and 9A6) or 45° C. (for 9A3 and 9A4) for 5 sec, 25 cycles in all.

It has been shown in the results that in the reaction with a denaturing temperature of 95° C., for all of the primers, whatever template contamination exists or not, positive results were obtained. However, at the denaturing temperature of 79° C., only for the templates containing contaminated amplified fragments, positive results were obtained. Negative results appeared for all of the long strand DNAs without contaminated fragments (Table 9).

TABLE 9
Detecting the contamination of amplified product fragment
at the denaturing temperature of 79° C.

| Prim- | Contami- | Quantity of contamination | | | | | | |
|---|---|---|---|---|---|---|---|---|
| er | nation of | of amplified products (wt/20 μL) | | | | | | |
| pair | template | 10 ng | 1 ng | 100 pg | 10 pg | 1 pg | 100 fg | 10 fg |
| 9A1 | None | − | − | − | − | − | − | − |
| | added | + | + | + | + | + | + | + |
| 9A2 | None | − | − | − | − | − | − | − |
| | added | + | + | + | + | + | + | ± |
| 9A3 | None | − | − | − | − | − | − | − |
| | added | + | + | + | + | + | + | + |
| 9A4 | None | − | − | − | − | − | − | − |
| | added | + | + | + | + | + | + | + |
| 9A5 | None | − | − | − | − | − | − | − |
| | added | + | + | + | + | + | + | ± |
| 9A6 | None | − | − | − | − | − | − | − |
| | added | + | + | + | + | + | + | ± |

+ indicates positive results;
− indicates negative results;
± indicates weak positive results.

Long strand DNAs can be melted at 95° C., a high denaturing temperature, but not denatured at 79° C. However, the contaminated fragments can be amplified to give positive results both at 95° C. and 79° C. The method of the invention can be used to discriminate clearly whether the templates in the DNA samples being detected are long strand DNAs or contaminated fragments brought about by the former amplified products.

### EXAMPLE 7

Application in Assay for the Discrimination of Genomic DNA and cDNA.

PCR method of the invention also has unique application in assays for the discrimination of genomic DNA and cDNA. For genomic DNA and cDNA, PCR reactions were performed with primer pairs relevant to the super-short products designed in example 6, 9A1-9A6. The template samples were subjected to following two PCR reactions:

(1) denaturing at a denaturing temperature of 95° C. for 15 sec, annealing and extending at 62° C. (for 9A1, 9A2, 9A5 and 9A6) or 45° C. (for 9A3 and 9A4), for 5 sec in the primary two cycles; then denaturing at 79° C., and annealing and extending at 62° C. (for 9A1, 9A2, 9A5 and 9A6) or 45° C. (for 9A3 and 9A4) for 5 sec, 25 follow-up cycles in all.

(2) Except the primary cycle number is one, the rests are the same as (1) Genomic DNA can be melted at 94-95° C. and not be denatured at 68-87° C., so it needs at least two high-temperature denaturing cycles to complete the amplification for genomic DNA, while it only needs one high-temperature denaturing cycle for the complementary DNA (cDNA), obtained from the reverse transcription of gene-specific reverse primer, to complete the amplification.

It has been shown in the PCR that, for genomic DNA, positive results appear in reaction (1) and negative results appear in reaction (2); whereas positive results appear in both reaction (1) and (2) for cDNA.

What is claimed is:

1. A method of polymerase chain reaction with ultra-low denaturing temperatures, including steps of:
(1) denaturing templates;
(2) annealing primers;
(3) extending and synthesizing complementary DNA strands by catalysis of DNA polymerase,
wherein the denaturing temperature of the template being in the range of 93-98° C. in primary two or three cycles, and 60-87° C. in the follow-up cycles.

2. The method of polymerase chain reaction according to claim 1, wherein the denaturing temperature of the template being in the range of 70-82° C.

3. The method of polymerase chain reaction according to claim 1 or claim 2, wherein the length of the amplified product is from 24 to 1,000 bases.

4. The method of polymerase chain reaction according to claim 1 or claim 2, wherein the length of the amplified product is from 40 to 150 bases.

5. Use of the method of polymerase chain reaction of claim 1 in the elimination of non-specific amplified products, characterized in that the difference between the denaturing temperature of the original template and that of the product is 7-28° C.

6. Use of the method of polymerase chain reaction in the elimination of non-specific amplified products according to claim 5 is from 10 to 20° C.

7. Use of the method of polymerase chain reaction of claim 1 in the exclusion of false-negative results, characterized in that the mismatching between the original template and the primer is from 1 to 5 bases, and the melting temperature of the product is 60-87° C.

8. Use of the method of polymerase chain reaction in the exclusion of false-negative results according to claim 7, characterized in that the mismatching between the original template and the primer is from 1 to 3 bases.

9. Use of the method of polymerase chain reaction in the exclusion of false-negative results according to claim 7, characterized in that the annealing temperatures of the primer and the template are in the range of 32-65° C., respectively.

10. Use of the method of polymerase chain reaction in the exclusion of false-negative results according to claim 7 or claim 8, characterized in that the annealing temperatures of the primer and the template are in the range of 46-58° C., respectively.

11. Use of the method of polymerase chain reaction of claim 1 in the detection of the contamination in the amplified products, characterized in that the templates are reacted at denaturing temperatures of 94-95° C. and 68-87° C., respectively, in primary two cycles.

12. Use of the method of polymerase chain reaction of claim 1 in the detection to discriminate genomic DNAs and cDNAs, characterized in that two PCR reactions are performed with template samples as follows:
(1) performing primary two cycles at the denaturing temperature of 94-95° C., and follow-up cycles at the denaturing temperature of 68-87° C.; and
(2) Performing primary one cycle at the denaturing temperature of 94-95° C., and follow-up cycles at the denaturing temperature of 68-87° C.
